(19) United States
(12) Patent Application Publication
(10) Pub. No.: US 2003/0124567 A1
(43) Pub. Date: Jul. 3, 2003

(54) Use of a leptospire protein preventing and/or diagnosing and/or treating animal or human leptospirosis

(76) Inventors: Genevieve Andre-Fontaine, Nort Sur Erdre (FR); Christine Branger, Vertou (FR)

Correspondence Address: ALSTON & BIRD LLP, BANK OF AMERICA PLAZA, 101 SOUTH TRYON STREET, SUITE 4000, CHARLOTTE, NC, 28280-4000, US

(21) Appl. No.: 10/203,253

(22) PCT Filed: Feb. 8, 2001
(86) PCT No.: PCT/FR01/00373

(30) Foreign Application Priority Data
Feb. 8, 2000 (FR) 00/01527

(51) Int. Cl. C12Q 1/68; G01N 33/554; G01N 33/569; C07H 21/04; C12N 1/21; C12N 15/74

(52) U.S. Cl. 435/6; 435/7.32; 435/69.3; 435/320.1; 435/252.3; 530/350; 536/23.72; 530/388.4

(57) ABSTRACT

The invention concerns the identification of a leptospire protein, polypeptides and antibodies, and their uses for prevention, diagnosis, therapy or experimentation. The invention concerns in particular the isolation, cloning and characterisation of a protein or fragments thereof, containing immunogenic epitopes against leptospiral serovar, in particular pathogenic liptospires. Said protein, polypeptides or peptides derived therefrom, and the corresponding antibodies are particularly useful for preventing, screening or detecting the presence of leptospires in biological samples, for diagnosing leptospirosis, or for treating said pathologies, in human and animal subjects.

The present invention relates to the identification of a leptospire protein, to polypeptides and antibodies, and also to the uses thereof in the diagnostic or therapeutic domains, the domain of prevention or the experimental domain. The present invention in particular describes the isolation, cloning and characterization of a protein, or fragments thereof, containing epitopes which are immunogenic with respect to multiple serotypes of leptospires, in particular of pathogenic leptospires. This protein, polypeptides or peptides derived therefrom, and the corresponding antibodies are particularly useful for screening for or for detecting the presence of leptospires in samples (biological or nonbiological), for diagnosing leptospiroses, or for the prevention or treatment of these pathological conditions, in humans and in animals.

Pathogenic leptospires are responsible for infectious diseases which affect humans and animals. Leptospiroses are among the zoonoses monitored by the WHO due to their worldwide distribution and the seriousness of the disease (lethal in 2 to 20% of diseases depending on the earliness of diagnosis and of treatment). In animals, the economic impact is considerable due to the consequences of the disease, which is responsible for reproductive disorders in breeding animals (ruminants or pigs) or for serious clinical signs which modify the health of pets, namely dogs, or that of animals used in sport, namely horses.

The importance of leptospiroses is in particular illustrated by two aspects: first of all, their impact in public health (hospitalizations, inability to work, sometimes deaths, implementation of vaccinations in the context of protection against occupational diseases in certain countries, including France); secondly, their economic impact (loss of production for breeding animals and cost of the vaccines used in many countries in order to combat this drop in production, emotional loss of pets and/or cost of vaccines currently available for dogs in virtually all countries throughout the world, and also financial loss due to impairment of sporting performances of horses).

It is particularly difficult to combat these infections given the bacteriological complexity of these microorganisms and the diversity of the reservoirs constituted by the animals of wild fauna, which carry, excrete and disseminate these microorganisms via their urine. The bacteriological complexity of these bacteria is such that, currently, two classifications coexist for these microorganisms: a serological classification and a genomic classification.

According to the serological classification, all the pathogenic strains have been classified in the same pathogenic species Leptospira interrogans, as opposed to the saprophytic strains found in water and classified in Leptospira biflexa. The species Leptospira interrogans is divided into more than 25 different serogroups, each of these serogroups being, itself, composed of several serotypes exhibiting among one another a sufficient number of common antigens for them to be grouped together. Some 220 different serotypes thus exist. This classification is established on the basis of the agglutinating antibody-inducing antigens; for this reason, it is still the only one used in the context of serological diagnosis of the infection.

According to the genomic classification, based on the study of genetic homologies, the pathogenic species Leptospira interrogans sl has been broken up into different species, termed genomospecies: Leptospira interrogans sensu stricto, Leptospira kirshneri, L. borgpetersenii, L. weilii, L. noguchii, L. santarosai, L. meyeri.

The clinical expression of leptospirosis is very polymorphic, depending on the infecting strains but also on the species.

Thus, humans, sensitive to leptospirosis, generally develop rapidly progressing acute forms with the liver, kidneys or lungs being affected. The earliness of diagnosis generally conditions the effectiveness of antibiotic treatment. The French vaccine directed against Icterohaemorrhagiae provides protection only against this serogroup considered to be the most pathogenic.

In dogs, the most sensitive animal species, vaccination against the two serogroups considered to be dominant in this species (Tcterohaemorrhagiae and Canicola) have been carried out. However, the vaccine only protects against these two serogroups and does not make it possible to avoid infection with a wild-type strain belonging to another serogroup. This explains the development of chronic disorders, which are still incorrectly identified by veterinary practitioners who consider that a dog vaccinated against “leptospirosis” cannot develop a chronic form related to infection with a wild-type strain.

In horses, which are greatly at risk due to the conditions under which they are kept, rare acute forms, but also uncharacteristic forms, such as drops in form and substandard sporting performances, appear. Some abortions in this species are also related to leptospirosis infection. Finally, a recurring eye ailment is attributed to the sequels of infection with leptospires. This ailment, which compromises the animal's future due to the blindness which it may cause, is, moreover, in France on the list of latent defects.

In ruminants and pigs, leptospirosis is essentially expressed through reproductive disorders, comprising abortions or infertility, these being disorders for which the economic weight may be estimated to be sufficiently great for certain countries to set up vaccination and/or eradication measures.

Given the importance and the diversity of the pathological forms, any suspicion of leptospirosis, whether it is an individual clinical case or a breeding stock problem, is generally the subject of experimental confirmation methods. Diagnosis (and screening) in two parts may thus be necessary, depending on the duration of progression of the disease. In acute cases, direct demonstration of the microorganism is primordial. In chronic cases, indirect demonstration via the serological response is generally sufficient in terms of diagnosis, but, in a second step, recognition of animals which are carrying microorganisms and are therefore excreters requires demonstration of the microorganism.

Currently, the direct demonstration of the pathogenic agent of the microorganism can be carried out essentially by two methods: isolation and PCR.

Bacteriological isolation is a very laborious method. The natural fragility of the leptospires means that the specimens treated in a limited period of time allow the possible isolation of a leptospiral strain. It should be noted that the probability of isolating a leptospire is generally low, given the requirements of these microorganisms and that, moreover, it may take several weeks to obtain a response given the slowness with which the cultures develop.

PCR has therefore proved to be a much more advantageous tool in terms of diagnosis. However, this method is currently only developed in humans, for which it is carried out using a blood specimen taken from the patient in the acute phase. Performing PCR as a diagnostic method on tissues such as products from abortion is still only at the experimental stage. With regard to screening for animals which excrete leptospires in their urine, a method which would be useful in animal rearing, it is not currently operational.

Serological diagnosis of the microorganism, consisting in demonstrating antibodies which reveal an infection, is generally carried out using microagglutination (MAT) which is currently the method of reference. The drawbacks of this method are related to the bacteriological heterogeneity of leptospires. The method requires the use of live microorganisms (the culturing of which is delicate) and is, moreover, based on demonstrating agglutinating antibodies induced by the surface antigens of leptospires, these being antigens which were used to classify the leptospires and therefore express the heterogeneity of these bacteria. This means that the use of serological diagnosis requires the patient's serum to be brought into contact with a live strain representative of each of the different serogroups, multiplying the manipulations for the same serum.

The present invention at this time makes it possible to resolve the drawbacks of the various screening, diagnostic or treatment methods described in the prior art. The present invention in fact describes the isolation of a leptospire protein bearing antigenic units common to multiple leptospiral serotypes, in particular serotypes of pathogenic leptospires. The present invention thus makes it possible to carry out tests for detecting or screening for the leptospires themselves or the infection which they cause, which are not restricted to particular serotypes. The present invention also makes it possible to carry out detection tests which are simpler than the MAT methods described in the prior art, since it does not require the culturing of microorganisms. The present invention also makes it possible to produce polypeptides, peptides and antibodies which can be used in effective vaccination approaches capable of inducing protection or an immune response against multiple pathogenic leptospiral serotypes. The present invention also describes nucleic acids, vectors, probes, primers and also recombinant cells which can be used for producing the polypeptides, peptides or proteins or for detecting leptospires or the products thereof in any test sample which is biological (for example biological fluids such as blood, plasma, urine, tissue, organs, cell culture etc.) or of another origin (such as, for example, a sample soiled with biological material, such as river water, stagnant water, drinking water, stored water used by companies such as slate quarries, market gardeners, etc., other types of drinks, milk, etc.).

A first aspect of the invention lies more particularly in the isolation and characterization of a protein which is common to the pathogenic leptospiral strains (or to some of them) and very immunogenic whatever the infected species under consideration. It is more particularly an approximately 32 kD protein, designated PPL, isolated from preparations of an Autumnalis strain, the structure of which comprises the internal sequence TFLPYGSVINYYGYVK (SEQ ID NO: 1).

Studying this sequence in the databases (Genbank and Swiss Prot) shows that this sequence has a partial identity with hemolysis associated protein 1 described by ML KIM, Korea University of Seoul, for the laiomer serotype of the Icterohaemorrhagiae group and a partial identity with the LipL32 protein (WO99/42478). The publication by Kim, however, makes absolutely no mention of the immunogenic and immunizing properties of the protein and describes no specific probe, antibody or primer. The LipL32 protein exhibits structural differences with PPL (for example Pro215-Thr) and it has in no way been demonstrated that cross protection can be obtained. In addition, the LipL32 protein is described as being a membrane protein, whereas PPL is also secreted. No other significant homology has been demonstrated.

A first subject of the invention lies in a polypeptide comprising the sequence SEQ ID NO: 1.

The term “polypeptide” denotes any molecule the primary structure of which is mainly composed of a series of amino acids. A polypeptide according to the invention may comprise up to 1000 amino acids for example. The term “protein” denotes, in the strict sense, a subgroup of polypeptides, comprising molecules of essentially natural origin. The term “peptide” denotes more particularly polypeptides which are small in size, typically 30 amino acids or less.

More particularly, the polypeptide according to the invention is a leptospire protein, in particular a membrane or secreted leptospire protein, more preferentially from a pathogenic leptospiral strain. The polypeptide or protein of the invention advantageously has an average molecular weight of between approximately 30 kDa and approximately 35 kDa, determined by gel electrophoresis.

A more particular subject of the invention consists of the PPL protein having a molecular weight of approximately 32 kD and comprising the sequence SEQ ID NO: 1.

It is more particularly a polypeptide comprising the sequence SEQ ID NO: 7 (represented in FIG. 3), preferably in soluble or secreted form, in particular residues 1-272 of SEQ ID NO: 7.

Another subject of the present invention lies in a polypeptide or peptide comprising the sequence of a protein of a pathogenic leptospire, of a membrane protein of a pathogenic leptospire or of a protein secreted by a pathogenic leptospire, or of a region of such a protein, and bearing one or more epitopes which are immunogenic with respect to several pathogenic leptospiral strains. The present invention in fact lies in the demonstration of polypeptides capable of inducing effective immune protection against several pathogenic leptospiral serotypes. Such polypeptides, or immunogenic peptides derived therefrom (alone or included in molecules which are larger or linked to carriers, etc.), constitute advantageous embodiments of the present invention, in particular in the form of a soluble or secreted polypeptide.

The term “epitope” is well known to those skilled in the art and denotes a sequence of continuous or spatially close amino acids, generally composed of 3 to 15 amino acids, recognized either by antibodies or by antigen receptors of T lymphocytes in combination with molecules of the class I or II major histocompatibility complex. Such epitopes can therefore induce the stimulation of a cellular or humoral immune response.

In this regard, a particular subject of the invention lies in an immunogenic fragment of a protein or of a polypeptide as described above. The fragments according to the invention advantageously comprise from 3 to 50 amino acids, more preferentially from 3 to 25 amino acids. The invention also relates to derivatives of fragments, polypeptides or proteins as described above, comprising, for example, sequence variations, for example one or more mutation(s), substitution(s), deletion(s) and/or insertion(s) of one or more residue(s). Preferentially, less than approximately 10% of the residues are modified in the variants of the invention, even more preferentially less than 5% of the residues.

The immunogenic nature of the polypeptides or peptides of the invention may be verified in various ways known to those skilled in the art. Thus, the polypeptides may be brought into contact with antibodies (or serum from infected individuals), the demonstration of antigen-antibody complexes indicating the capacity of the polypeptides of the invention to bear immunogenic epitopes or fragments.

The polypeptides, proteins and peptides of the invention may be produced according to various methods, such as by the recombinant pathway or artificial synthesis, or combinations thereof. The polypeptides, proteins and peptides of the invention may also be modified so as to comprise unnatural residues, chemical modifications and/or labels (fluorescent, radioactive, enzymatic, etc.).

Another subject of the invention relates to antibodies which recognize a protein, a polypeptide or a peptide according to the invention. They are preferentially anti-leptospire antibodies which bind an epitope present in a protein, a polypeptide or a peptide according to the invention. The antibodies of the invention are preferentially specific for leptospires, in particular pathogenic leptospires, although weaker (or nonspecific) binding may be observed experimentally with other antigens.

The antibodies according to the invention may be polyclonal or monoclonal antibodies. They are generally produced by immunizing an animal with a protein, a polypeptide or a peptide according to the invention and recovering the serum (in order to obtain the polyclonal antibodies), or thymus or spleen cells in order to produce monoclonal antibody-producing hybridomas.

The antibodies according to the invention are more preferentially antibodies which recognize the PPL protein of sequence SEQ ID NO: 7 as defined above, or a fragment thereof, preferably in soluble or secreted form.

The antibodies of the invention advantageously have the ability to recognize at least two pathogenic leptospiral strains belonging to different serogroups, more preferentially at least 3 pathogenic leptospiral strains, in particular belonging to different genomic species.

According to a particular embodiment, the antibodies according to the invention are polyclonal antibodies prepared by immunizing an animal with a PPL protein as defined above or an immunogenic fragment of this protein. The animal may be a rodent (rat, mouse, rabbit, gerbil, hamster, guinea pig, etc.), a primate, a pig, a horse, a bovine, a bird, etc. The polyclonal antibodies are generally recovered in the serum of the immunized animals or the eggs, according to protocols known to those skilled in the art.

According to another particular embodiment, the antibodies according to the invention are monoclonal antibodies prepared from hybridomas obtained by fusion between an immortalized cell (for example a myeloma) and an antibody-producing cell taken from an animal immunized with a PPL protein as defined above or an immunogenic fragment of this protein. The animal may be a rodent (rat, mouse, rabbit, gerbil, hamster, guinea pig, etc.), a primate, a pig, a horse, a bovine, a bird, etc.

The antibodies of the invention, in particular the monoclonal antibodies, may also be humanized, i.e. artificially modified so as to comprise regions of heavy or light chains of human origin.

The invention also relates to fragments or derivatives of such antibodies, for example Fab fragments, F(ab′)2 fragments, ScFv (single-chain antibodies), etc.

As will be developed in the remainder of the text, the antibodies of the invention may be used, for example, for detecting pathogenic leptospiral strains, or products secreted by these same leptospires, in test samples (in particular blood or urine specimens), or for inducing (emergency) protection against leptospiral infections.

The antibodies which are preferred for the purpose of the invention are antibodies, in particular monoclonal antibodies, capable of recognizing the PPL protein at various stages of maturation (for example in pro, mature and/or secreted form). As illustrated in the examples, such antibodies have improved properties for diagnosis and/or therapy.

Another subject of the invention lies in any nucleic acid encoding a polypeptide, peptide or protein as defined above. The nucleic acids may be DNAs or RNAs, in particular recombinant, genomic, synthetic or semi-synthetic DNAs or else mRNAs, or fragments or derivatives thereof. The nucleic acids may be obtained from libraries, cloned from leptospire bacteria (in particular by PCR), produced by artificial synthesis using nucleic acid synthesizers, or else prepared using combinations of these methods (enzymatic digestion, ligations, cloning, modifications, etc.). The nucleic acids may also be modified so as to improve codon usage, remove cryptic promoters, reduce secondary structures, etc.

In this regard, the present application describes the cloning of the ppl gene encoding the PPL protein as described above. The complete nucleic acid sequence of this gene is represented in FIG. 2 with its histidine label (SEQ ID NO: 6). Any fragment or derivative of this gene or of its sequence can be prepared using the conventional techniques of those skilled in the art. Any sequence which hybridizes this gene, or the strand complementary thereto, and which is capable of encoding a protein, a polypeptide or a peptide as defined above constitutes another particular subject of the invention. It is advantageously nucleic acids from leptospire, hybridizing under high stringency conditions, in particular the stringency conditions described in the examples, with all or part of the sequence SEQ ID NO: 6.

The invention also relates to vectors comprising a nucleic acid as defined above. It may be a vector of plasmid, cosmid, episome, artificial chromosome, phage, virus, etc. type. It is more preferentially a plasmid, for example a plasmid which replicates or integrates in bacteria or eukaryotic cells (yeasts, mammalian cells, bird cells, etc.) or a recombinant virus, in particular a defective virus (such as an adenovirus, a retrovirus, an AAV, a vaccinia virus, HSV, etc.).

The plasmid vectors may be prepared by conventional techniques using commercially available plasmids, such as pUC, pBR, pCN, etc.

The viral vectors may also be produced according to methods described in the prior art, and in particular using encapsidation cells. For adenoviruses, the vectors are generally defective for all or part of the E1 region, and may be produced in the 293 line or other cells described in the prior art. Other regions may, of course, be deleted from the genome, such as E2, E4 and/or E3 for example.

With regard to effective recombinant retroviruses, they are generally deleted of the gag, pol and env genes, and produced in lines such as PsiCRIP. With regard to AAVS, they may be produced in various lines, in the presence of a helper adenovirus and of a plasmid carrying the rep and cap functions. It is understood that any other technique for producing recombinant viruses may be used, without departing from the present application.

The invention also relates to any recombinant cell comprising a nucleic acid or a vector as defined above. The recombinant cell may be a bacterium (for example a strain of E. coli), or a eukaryotic cell, in particular a yeast, a mammalian cell, etc. The recombinant cells may be used in particular for producing the polypeptides of the invention, and also as models for searching for compounds capable of neutralizing or antagonizing the activity of the PPL protein.

The invention also relates to any nonhuman mammal or any bird comprising a nucleic acid as defined above in its cells. Advantageously, these mammals are obtained by homologous recombination. Such mammals (rodents, canines, rabbits, goats, pigs, etc.) can in particular be used for producing the polypeptides of the invention and for identifying compounds for therapeutic or vaccinal purposes, for example.

Another subject of the present invention lies in nucleotide probes and/or primers which can be used for detecting and/or amplifying leptospire nucleic acids, and in particular for detecting the presence of a pathogenic leptospiral strain, or of a product secreted by this leptospire, in a test sample (in particular a biological product or contaminated with a biological product).

The nucleotide probes according to the invention advantageously comprise all or part of a nucleic acid as defined above. They are preferentially single-stranded and may be labeled, for example by radioactive, enzymatic, fluorescent, chemical, etc. labeling. A probe according to the invention preferentially comprises a sequence complementary to all or part of the nucleotide sequence SEQ ID NO: 6 (FIG. 2). The probes of the invention may be used for detecting the presence of a pathogenic leptospiral strain, or of a product secreted by this leptospire, in any sample, in particular a biological sample. In fact, preferentially, they (i) are specific for pathogenic leptospiral strains and (ii) react with different serotypes and different genomic species of pathogenic leptospires. A preferred probe according to the invention comprises the complete sequence of the ppl gene (FIG. 2), or a fragment thereof comprising the sequence encoding SEQ ID NO: 1, preferably a fragment greater than 100 bases, more preferentially less than approximately 500 bases.

The nucleotide primers according to the invention are oligonucleotides, generally less than 40 bases in size, comprising the sequence of at least part of a nucleic acid as defined above. The primers can be used for amplifying leptospire nucleic acids, in particular for amplifying the ppl gene, or part of this gene, for example by PCR.

Such specific (and degenerate) primers are for example:

Primer 1 (SEQ ID NO: 3)

5′ ATAAGAATGCGGCCGCATGAAAAAACTTTCGATTTTGGC-3′

This primer has a 16 bp sequence in 5′ to allow the creation of a NotI restriction site which is of use for inserting the amplification product into an expression vector.

Primer 2 (SEQ ID NO: 4)

5′ CCGCTCGAGCTTAGTCGCGTCAGAACGCAGC-3′

This primer has a 9 bp sequence in 5′ which allows the creation of an XhoI site which is of use for inserting the PCR fragment into a vector.

The invention more particularly relates to any pair of primers which allows a region of the ppl gene as defined above to be amplified. Preferentially, the amplified region comprises at least 50 bp, preferably at least 150 bp.

The probes, primers or oligonucleotides of the invention preferentially have the ability to hybridize specifically with all or part of the genome of pathogenic leptospiral strains. The hybridization is termed specific when the probe or the oligonucleotide hybridizes, under high stringency conditions, with said genome and not, or hardly at all, with the genome of other bacterial species, in particular nonpathogenic leptospires. In particular, the hybridization is therefore termed specific when the specific signal/background noise differential is sufficiently great to be detected. A hybridization under high stringency conditions is, for example, a hybridization in 1% SSC, 0.1% SDS at 39° C.

The probes, primers or oligonucleotides of the invention are preferentially complementary to at least one region of the ppl gene. The complementarity is generally complete, so as to provide a better selectivity of hybridization. However, some mismatches can be tolerated. These probes or oligonucleotides may be synthesized by any technique known to those skilled in the art, for example by cleavage from the nucleic acids described above, or by artificial synthesis, or by combining these techniques. The probes and primers are of particular use for detecting the presence of pathogenic leptospiral strains and/or diagnosing leptospiroses, as will be detailed below.

Various teams work on improving the antigens for indirect diagnosis of leptospirosis, the aim being to produce operational methods which can be readily used in particular in nonindustrialized countries, which are major victims of these pathological conditions. The products currently provided on the market are complex extracts of leptospire cultures. This complexity reproduces that of the live strains used in the MAT and does not therefore provide any substantial improvement in terms of reproducibility and practicability.

The demonstration of antibodies directed against a pathogenic strain-specific antigen is therefore particularly advantageous. The present invention may thus be used from a diagnostic, vaccinal, therapeutic and/or experimental point of view.

In terms of indirect diagnosis, the polypeptides or peptides or the PPL protein of the invention, given its presence in, or its secretion by, the pathogenic strains, may be used as a purified protein antigen capable of demonstrating the antibodies produced during infection with a pathogenic strain, regardless of the serogroup to which it belongs. This therefore allows the experimental diagnosis of leptospirosis to be carried out in many laboratories which have ordinary equipment (for example ELISA or dot), whereas the MAT has a requirement for maintaining live cultures and, as a result, the standardization thereof is very difficult.

A particular subject of the invention therefore lies in the use of a polypeptide, peptide or protein as defined above, for detecting the presence of anti-leptospire antibodies in a biological test sample (in particular a biological sample such as blood, serum, urine, tissue, etc., or a nonbiological sample such as water, drink, etc.). Another subject of the invention lies in a method for detecting the presence of anti-leptospire antibodies in a test sample, comprising bringing this sample (or a dilution) into contact with a polypeptide, peptide or protein as defined above, and demonstrating the formation of antigen-antibody complexes.

In terms of direct diagnosis, the antibodies (or antibody fragments or derivatives) according to the invention, in particular the monoclonal antibodies of the invention, allow direct demonstration of the pathogenic agent, or of secreted products, present in a test sample (such as a specimen or another type of sample such as water). The demonstration may be performed by any immunological method known to those skilled in the art, such as by capture ELISA, RIA, direct or sandwich assays, etc., or by immunological revelation of this protein in the pathological or non-pathological specimens.

Another subject of the invention therefore lies in the use of an antibody (or antibody fragment or derivatives) according to the invention, in particular of one or more monoclonal antibodies of the invention, for detecting the presence of a pathogenic leptospiral strain, or of the secreted products thereof, in a sample, in particular a biological sample. Another subject of the invention lies in a method for detecting the presence of a pathogenic leptospiral strain, or of the secreted products thereof, in a sample, in particular a biological sample, comprising bringing this sample (or a dilution) into contact with antibodies (or antibody fragment or derivatives) according to the invention, and demonstrating the formation of antigen-antibody complexes.

For these uses, the polypeptides, peptides, proteins and antibodies may be used in soluble form or immobilized on solid or semi-solid supports of the filter, silica, glass, plate, bead, etc. type. The use in immobilized form advantageously makes it possible to simplify the detection or experimental diagnosis of leptospirosis. These compounds may also be labeled, for example with fluorescent, enzymatic, biological or radioactive labels. As indicated above, antigen-antibody complexes may also be revealed using an additional labeled antibody, or according to known immunological techniques (ELISA, RIA, sandwich, capture, etc.).

Another detection (or screening) method according to the invention lies in bringing a test sample into contact with a nucleotide probe of the invention, and demonstrating hybridization between said probe and said sample. More preferentially, the test sample is treated beforehand so as to make the nucleic acids which it contains accessible to a hybridization reaction. The treatment may consist in rupturing the cell membranes, for example by chemical (detergent) and/or mechanical (ultrasound, freezing-thawing, etc.) treatment. In a particular embodiment, the sample, in particular the biological sample, thus treated is subjected to an amplification reaction using primers of the invention, prior to the hybridization reaction with the probe. The amplification may be carried out under conventional conditions. The hybridization may be carried out on supports, on which the probe is immobilized (filters, glass, silica, etc.). The stringency conditions for the hybridization may be adjusted by those skilled in the art, by adjusting the temperature and/or the salinity of the media.

The invention also relates to a kit for carrying out the methods of the invention, comprising a probe or an oligonucleotide or a pair of primers as described above. The kits of the invention advantageously comprise the reagents suitable for an amplification and/or hybridization reaction and, optionally, a support for such reactions (filters, membranes, chips, etc.).

The present invention can also be used from the therapeutic and preventive point of view. In fact, although the pathogenic mode of action of the PPL protein has not yet been defined, the present application shows that it is capable of inducing protection. Administration of the antibodies directed against this protein, whether they are of polyclonal or monoclonal origin, makes it possible to neutralize the pathogenic impact of the leptospires in the individual during progression, and therefore to improve the prognosis for this disease. Similarly, administration of a polypeptide, peptide or protein of the invention, optionally in inactivated form, or of a corresponding nucleic acid or vector, makes it possible to induce a protective immune response against these infectious agents or their effects.

In this respect, the invention relates to immunogenic or vaccinal compositions comprising one or more polypeptides, peptides, proteins, antibodies, nucleic acids or vectors as described above. These compositions are particularly advantageous since they make it possible to induce an immune response or immune protection against various pathogenic strains, in particular against several different pathogenic serogroups.

In terms of protection, it is considered, in the prior art, that only whole bacteria are capable of providing the protective antigens and, moreover, that no cross protection exists between different serogroups. This means that a vaccine must be composed of a suspension, or more exactly of suspensions added together, of bacteria representative of each of the serogroups necessary for the protection of the species for which the vaccine is intended. Given the great diversity of the serogroups, a choice is necessary, which depends on the species and the epidemiological conditions. Thus, the usual vaccines used in dogs combine a suspension of inactivated bacteria of the Icterohaemorrhagiae serogroup and one of the Canicola serogroup. Vaccines used in pigs and ruminants in the United States combine suspensions of serotypes belonging to the serogroups: Icterohaemorrhagiae, Pomona, Grippotyphosa, Canicola and Sejroë (hardjo serotype) for example. The vaccine for human use sold in France at the current time comprises 2 serotypes of the same serogroup Icterohaemorrhagiae, etc.

The problem with these vaccinal preparations is their failure in terms of total protection against leptospirosis. A vaccinated individual for whom the vaccination is maintained according to the methods prescribed by the vaccine producers is protected against the infection and/or the disease induced by a wild-type strain belonging only to the serogroups present in the vaccine. Protection is therefore only provided (and even so this protection may be incomplete) against infection with one or more given serogroups.

Given the basic premise that it is impossible to induce cross protection between the different serogroups, the state of the art does not therefore make it possible to confer overall protection against leptospirosis, whatever the infecting serogroup (or even whatever the genomospecies) to which the individual is exposed.

The present invention at this time shows that it is possible to induce an effective immune response against pathogenic leptospires without using whole bacteria. The present invention also shows that it is possible to generate cross protection between several leptospirosis serogroups.

Thus, in a first step, it has been demonstrated that the presence of whole bacterial bodies is not essential for the induction of homologous protection. The expression “homologous response or protection” is intended to mean the protection induced by a preparation consisting of the leptospires belonging to the same serogroup as the leptospires used in the challenge performed on a sensitive laboratory animal and making it possible to assess the authenticity of the protection conferred. This was demonstrated by immunizing animals with a total leptospiral extract obtained by rupturing the bacteria.

The second step has been to demonstrate that total extracts of different serogroups make it possible to induce heterologous protection within the species L. interrogans sl. Reference is made to heterologous protection when the challenge is performed with a strain which does not belong to the serogroup used for the immunization. This was demonstrated by immunization carried out with total extracts of cultures of the serogroups Autumnalis, Canicola or Icterohaemorrhagiae, followed by challenges using Icterohaemorrhagiae or Canicola strains, the virulence of which is maintained in the laboratory.

The third step has been to demonstrate that the antigen(s) responsible for this cross protection is (are) absent from, or expressed very little by, the saprophytic species and, optionally, present in strains of different serogroups but also belonging to different genomic species: a total extract of L. biflexa used did not make it possible to protect animals against a challenge.

The fourth step has been to define the nature of the antigen(s) responsible for this cross protection. Purified lipopolysaccharide (LPS) extracts were prepared by the Westphal method of extraction with phenol-water under hot conditions. This preparation method makes it possible to obtain the pure lipopolysaccharide (LPS) fraction. The residual extract contains the proteins (simple or complex), but also residual LPS. We have demonstrated that the pure LPS extract is responsible for a powerful protective, but exclusively homologous, effect. This clearly confirms the prior data regarding the lack of cross protection between serogroups. In fact, the LPS corresponds to the outer antigenic structures responsible for the production of agglutinating antibodies used as a basis for the serological classification, but also responsible for the protective effect of the vaccinal preparations. It was therefore necessary for a vaccine to be capable of inducing the production of agglutinating antibodies at significant levels in order to protect an animal.

The fifth step has been to monitor the ability of a protein extract of an Autumnalis or Canicola strain, purified of any trace of LPS, to induce homologous protection in the same way as the purified LPS, but also to induce protection against a heterologous challenge observed during the previous tests. This was carried out by immunization with a protein extract obtained by a chloroform/methanol extraction performed on the interface of a phenol/water extraction which had allowed the prior purification of LPS, followed by a homologous or heterologous challenge depending on the case.

The sixth step has been to define the molecular weight range of the proteins which are effective in the heterologous protection. This point was demonstrated by the induction of significant protection induced after immunization of animals with the gel electrophoresis band comprising the proteins segregating around 32-34 kD of a protein extract extracted from Autumnalis.

During the seventh step, it has been possible, using the Biorad Prep-cell, to separate various proteins segregating in this range, in order to carry out the sequencing thereof. Three bands were the subject of the sequencing study, a 32 kD protein and two 34 kD proteins. The NH₂-terminal sequences have been defined, as has an internal sequence of about twelve amino acids corresponding to the major peak (peak 14) obtained by HPLC. The internal sequence of the 32 kD protein has been defined as being the sequence SEQ ID NO: 1. This protein has been designated PPL.

In an eighth step, degenerate nucleotide probes have been constructed, the ppl gene has been cloned, and recombinant PPL protein has been produced, which has allowed the production of monoclonal antibodies. The ppl gene has been inserted into a nonreplicative human adenovirus and effective protection against challenges performed on gerbils has been observed after administration of these recombinant viruses, in particular as vaccines.

These results demonstrate that, while the lipopolysaccharide antigens are clearly the carrier of effective protection with respect to a homologous infection, which is the starting point for constituting the vaccinal preparations currently used both in humans and in animals, the PPL protein, used alone, is capable of inducing cross protection between different serogroups of the species Leptospira interrogans ss (Icterohaemorrhagiae, Canicola, Autumnalis). Heterologous protection is also induced between different genomic species of the traditional species Leptospira interrogans sl (here L. borgpetersenii and interrogans ss), whereas it is absent from the traditional saprophytic species Leptospira biflexa.

Complementary results have made it possible to validate the heterologous protective effect of PPL and to show that this protein is not necessarily a membrane protein, but exists in soluble or secreted form (mature or immature form).

The present invention therefore relates to the use of the PPL protein, of synthetic or natural fragments thereof or of derivatives thereof whether they are used alone or combined with other proteins or lipopolysaccharide fractions derived from leptospires or with adjuvants of immunity whatever their nature, as a vaccinal preparation for veterinary or human use, in particular for preparing a composition intended to induce an immune response against different serotypes of pathogenic leptospires.

A subject of the invention is also a vaccine comprising a polypeptide or a nucleic acid as defined above.

A subject of the invention is also a composition comprising one or more antibodies as described above, in particular for inducing protection.

The immunogenic compositions or vaccines of the invention may be used in injectable or per os or transcutaneous form, for example in combination with vehicles which are acceptable from a pharmaceutical or veterinary point of view, or with adjuvants. The amounts of immunogen administered, and the frequency or number of the administrations, may be adjusted by those skilled in the art depending on the individual, on the state of progression at which the injection is given, etc. Typically, one or two injections, 1 month apart, are given with 10⁴ to 10⁹ pfu of recombinant virus according to the invention, or with 50 to 1000 μg of protein, polypeptide, peptide or antibody, at least. Additional injections may be envisioned, or larger amounts may be administered, in particular in the case of oral administration. Specific immunization protocols comprise, for example, two intramuscular injections, given three weeks apart, of a preparation of recombinant adenovirus comprising 10⁹ particles of adenovirus (CPED50) expressing a PPL polypeptide. It is understood that the adenovirus (or the virus) used may express other polypeptides in addition to the PPL polypeptides, or may be used in combination with other adenoviruses (or viruses) expressing other polypeptides (immunomodulators, immunogenic peptides, etc.).

The immunogenic compositions or vaccines of the invention in particular have the following advantages:

Effectiveness: induction of cross protection avoiding accumulation of different antigenic preparations in order to prevent infection with the various infecting strains present in a given country for a given species.

Innocuity: the use of this purified protein (or polypeptides, antibodies and nucleic acids) avoids introducing, into the vaccinated individual, many antigens which are unnecessary for the protection, and therefore at the very least useless, or even dangerous, when used repeatedly. It is thus thought that recurrent uveitis in horses (possibly resulting in blindness of the animal) is induced by a post-infectious immune response due to leptospire proteins having a composition close to certain structural proteins of the horse's eye. Moreover, it appears that, in humans, repeat vaccinations are frequently less well-tolerated over time.

Compatibility with prophylactic sanitary measures: currently, it is impossible to differentiate the post-vaccination antibodies from the post-infection antibodies, the production of agglutinating antibodies being obtained in both cases. This vaccine used in the large production species (there is not one currently in France, and the effectiveness of those which exist in foreign countries is difficult to demonstrate) would make it possible to implement medical prophylaxis, allowing, despite everything, the wild-type infection to be screened and therefore livestock to be labeled free of infection, this labeling increasing the value for export.

Limitation of the use of antibiotics in animal rearing: the lack of possibilities for eradication of leptospires (maintained in the environment by many species of animals forming the breeding ground of wild-type fauna) currently makes it necessary to implement antibiotic treatment extended to the entire herd when clinical disorders linked to leptospiral infection are recognized. This therefore contributes to the intensive use of antibiotics in animal rearing, the use being controversial in terms of consequences for public health.

The industrial production of such a vaccine could, finally, make it possible to simplify vaccine production due to the fact that the antigenic composition of the vaccinal preparation does not need to be adapted, firstly, to the species and, secondly, to the epidemiological conditions specific for the geographical regions where it would be applied.

The invention also relates to the polypeptides, proteins and peptides as defined above, in attenuated form, i.e. conserving the immunogenic properties and essentially free of other biological activity.

The invention also relates to the use of the nucleic acids or vectors as described above, for producing, in vitro or ex vivo, the polypeptides, proteins and peptides of the invention, whatever the methods of genetic recombination used: transgenic animals, bacteria, eukaryotic cells, plant cells, plasmids, viruses, etc. The techniques of producing recombinant proteins are well known to those skilled in the art and may be applied to the present invention (strong promoters, inducible promoters, termination signals, transfection techniques, etc.).

The present invention will be described in detail with the aid of the following examples, which should be considered to be nonlimiting illustrations.

### LEGEND TO THE FIGURES

FIG. 1: Restriction map of plasmid pET-29b-ppl.

FIG. 2: Nucleotide sequence of the gene of the recombinant PPL protein (SEQ ID NO:6).

FIG. 3: Sequence of the recombinant PPL protein (280aa, SEQ ID NO:7).

FIG. 4: Nucleic acid sequence of the 841 bp ppl PCR fragment (843 bp, SEQ ID NO:5).

FIG. 5: Immunoblot with two anti-PPL monoclonal antibodies. Demonstration of several reactive bands by comparison with an anti-autumnalis rabbit polyclonal antibody.

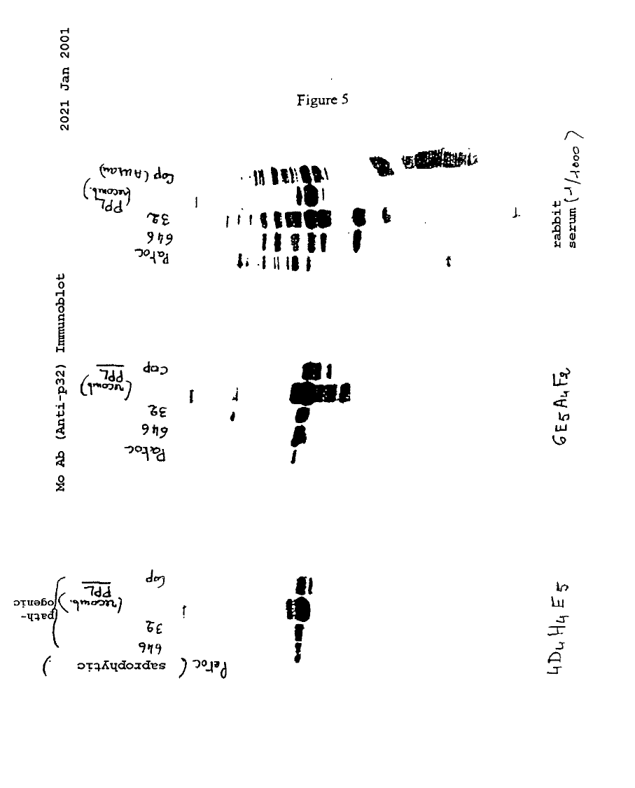

FIG. 6: Presence of the ppl gene in various genomic species.

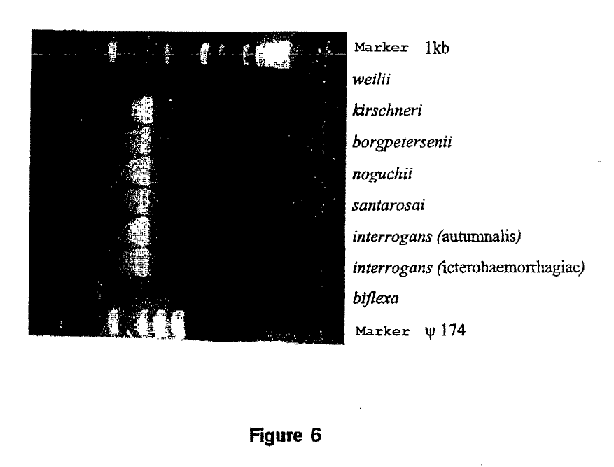

### EXAMPLES

1. Materials and Methods

Challenge in Gerbils:

The challenge is performed with an intraperitoneal injection of 0.5 ml of an inoculum of a subculture of less than 3 weeks of the pathogenic strain canicola. This subculture (prepared in medium base EMJH+10% specific enrichment+1% rabbit serum) is obtained by subculturing a re-isolation obtained after passage on a gerbil (cf. procedure for maintaining virulence). The subculture, with a titer of 82 TU (turbidimetry) after filtration at 0.8 μm, is diluted to 10⁻⁵ in sterile buffer medium (base EMJH).

Control of Strain Virulence: B2ML

The pathogenic capacity of the leptospiral strains of known pathogenic capacity (Icterohaemorrhagiae and Canicola) is controlled once or twice a year on gerbils. The animals receive a culture which has been subcultured, within the preceding three weeks, from an isolation carried out on ground material from the liver and kidney of a gerbil which died during the previous challenge.

A dose of 0.5 ml at 10 to 20 TU, injected intraperitoneally, induced death of the animals within a period of 3 to 7 days. Pathogenic strain: Canicola canicola (origin Institut Pasteur).

Materials:

The restriction enzymes and also the other enzymes required for the various DNA modifications were supplied by Roche Diagnostics and New England Biolabs Inc., the Taq polymerase (high fidelity) for the polymerase chain reactions was supplied by Gibco-BRL. All these enzymes were used according to the manufacturers' recommendations.

The following Escherichia coli strains were used:

DH5α™ competent cells (18265-017, Life Technologies™): Fφ80/acZΔM15 Δ(lacZYA-argF)U169 deoR recA1 endA1 hsdR17(r_{K}⁻, m_{K}⁺) phoA supE44 λ⁻ thi-1 gyrA996 relA1

BL21(DE3) competent cells (200131, stratagene): E. coli BF⁻ dcm ompT hcdS (r_{B}⁻m_{B}⁻) gal (DE3)

BJ5183 (Hanahan D. J. Mol. Biol. 1983. 1666. 557-580)

2. Identification and Isolation of an Immunogenic Leptospire Protein

Protein fractions were prepared from a total leptospire extract, by chloroform/phenol/water extraction, according to the method of Auran (N. E. AURAN, R. C. JOHNSON and D. M. RITZI), Isolation of the outer sheath of Leptospira and its immunogenic properties in hamsters, Infect. Immun. 1972, 5, 968-975). These fractions were prepared as a function of their molecular mass by polyacrylamide gel electrophoresis.

A first experimental protection test using the protein fractions obtained showed that it was possible to obtain incomplete but real protection after immunization of gerbils with electrophoresis gel bands excised by isolating the various protein bands as a function of their various molecular masses: example, 41-43 kDa region, etc. However, the extract used contained “LPS”, including in the electrophoretic regions in which it is not directly detectable, but its contaminating presence is observed on the immunized animals. Now, LPS is already known to be the carrier (essential if not unique) of immunity against leptospires.

Moreover, the antigenic characteristics which differentiate L. interrogans from L. biflexa were studied: these differences are essentially in the 21, 32, 34, 38, 41, 100 and 110 kDa bands.

In a second step, the protective capacity of the 26-31 and 31-34 bands of 2 leptospiral serotypes of the pathogenic species (Canicola strains and Autumnalis 32 strain) were therefore tested, and compared to the possible protection induced by a strain of the nonpathogenic species L. biflexa strain Buenos Aires.

This experiment allows us to conclude that:

The protective antigen is not present in the saprophytic species L. biflexa (no survival of the gerbils having been immunized with this antigen prepared as total extract as previously, from the 10^{th} day, the controls all being dead at the 8^{th} day), which means that none of the antigens which are common between interrogans and biflexa is protective (or it exists in too small an amount in biflexa).

The homologous (canicola) protection provided by the two bands 26/31 and 31/34 is complete.

Heterologous protection exists for vaccinated gerbils

Autumnalis 32: at the 21^{st} day

2/10 survive for the 26/31 band

4/10 survive for the 31/34 band.

However, the serological controls performed on the animals before challenge showed that those which had received the supposedly exclusively protein bands, such as the 31-34 band, had in fact been partially immunized with LPS. In fact, and subsequent experiments showed this, LPS, which is very immunogenic, diffuses more toward the high molecular weights by electrophoresis as the quantitative charge of the extract subjected to migration increases, such as the case of the preparative electrophoresis used for the gerbil immunizations.

Methods for separating the LPS and the proteins were therefore refined in order to remove the risks encountered in the previous experiment. Thus, it is possible to obtain extracts (9/1 phenol/water v/v, and then extraction with 2/1 chloroform/methanol v/v carried out on the phenol phase of the previous extraction) which contain no trace of the lipopolysaccharide fractions.

New immunizations were then carried out before challenge, the aim of which was to test the protective capacity of the protein fractions versus that of the LPS fractions now correctly separated.

For this, gerbils received:

Icterohaemorrhagiae protein extract

Icterohaemorrhagiae LPS

Canicola protein extract

Canicola LPS.

Each batch was divided into two and, as a result, received either a homologous challenge or a heterologous challenge.

This experiment allowed us to conclude that:

The protection with LPS is strictly homologous (no survival of the animals immunized with heterologous LPS).

Heterologous protection carried by the purified proteins exists: 6/6 of the gerbils having received the icterohaemorrhagiae proteins are still alive one month after the challenge with canicola.

Another batch of gerbils received a canicola protein extract before challenge with icterohaemorrhagiae. On the 5^{th} day of the challenge, the 20 controls are dead, 12/17 vaccinated are alive, and 9 are one month later.

This made it possible to complete the previous experiment on the heterologous protein protection, despite the difficulties in controlling a challenge performed with icterohaemorrhagiae.

Therefore, the significant protection obtained with the 31/34 extract cannot be due to traces of LPS, the latter having shown its strictly homologous protective capacity in subsequent experiments.

The proteins included in the 31/34 region were therefore separated using the Prepcell (Biorad), which made it possible to demonstrate the presence of at least two proteins, of 32 kD and 34 kD. Subsequent studies showed that the 32 kD protein (designated PPL) is, by itself, responsible for the protections observed.

A 32 kDa protein fraction of Leptospira interrogans autumnalis was isolated and purified by electrophoresis. Sequencing the PPL protein allowed us to obtain a PPL peptide minisequence of 16aa: TFLPYGSVINYYGYVK (SEQ ID NO: 1). Alignment of this minisequence with the Genbank databank gave 93% homology with a 29.613 kDa Hapl protein, the gene of which consisted of 819 bp (origin Leptospira interrogans lai).

3. Study and Expression of the ppl Gene

The genomic DNA of Leptospira interrogans serotype autumnalis was prepared by standard protocol. This DNA was digested successively with 4 restriction enzymes: BglII, DraI, EcoRI and HindIII. They were chosen as a function of the restriction map for the hap1 gene. This digested DNA was subjected to electrophoresis and it was then transferred onto a Hybond™-N⁺ membrane (Amersham). The hybridization was carried out with a degenerate nucleic acid probe synthesized from the peptide minisequence.

ppl probe (SEQ ID NO: 2):

5′-GTNATHAAYTAYTAYGGNTAYGTNAAAR-3′

This probe was radiolabeled with [γ-³²P]dATP. The hybridization was carried out at 39° C. overnight in hybridization buffer. The nylon membrane was then washed with 1% SSC (sodium chloride sodium citrate, pH 7) with 0.1% of SDS, at 39° C. The membrane with the hybridized probe was exposed on a Kodak film at −80° C.

3.1. Preparation of Specific Primers

Two suitable nucleotide primers were chosen from the ppl nucleotide sequences, in order to carry out PCRs on the genomic DNA of Leptospira interrogans serogroup Autumnalis. The sequence of the primers is given below:

Primer 1

5′-ATAAGAATGCGGCCGCATGAAAAAACTTTCGATTTTGGC-3′(SEQ ID NO: 3)

This primer has a 16 bp sequence (underlined) in 5′ so as to allow the creation of a NotI restriction site of use for inserting the amplification product into an expression vector. A preferred primer therefore comprises the sequence SEQ ID NO: 3, residues 17-39.

Primer 2

5′-CCGCTCGAGCTTAGTCGCGTCAGAAGCAGC-3′(SEQ ID NO: 4)

This primer has a 9 bp sequence (underlined) in 5′ which allows the creation of an XhoI site of use for inserting the PCR fragment into a vector. Another primer according to the invention comprises the following sequence:

5′-TTACTTAGTCGCGTCAGAA-3′ (SEQ ID NO: 8)

The PCR was carried out on a Perkin Elmer Cetus thermocycler under standard conditions with the primers mentioned above. 30 cycles were performed, each cycle consisting of the following program: denaturation (15 sec at 94° C.), hybridization (30 sec at 61° C.) and elongation (90 sec at 72° C.). The genomic DNA of Leptospira interrogans serogroup autumnalis was used as matrix.

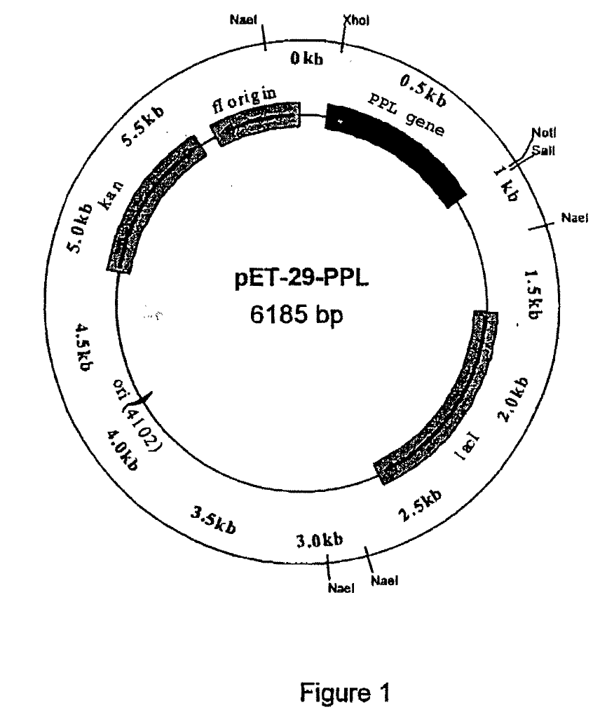

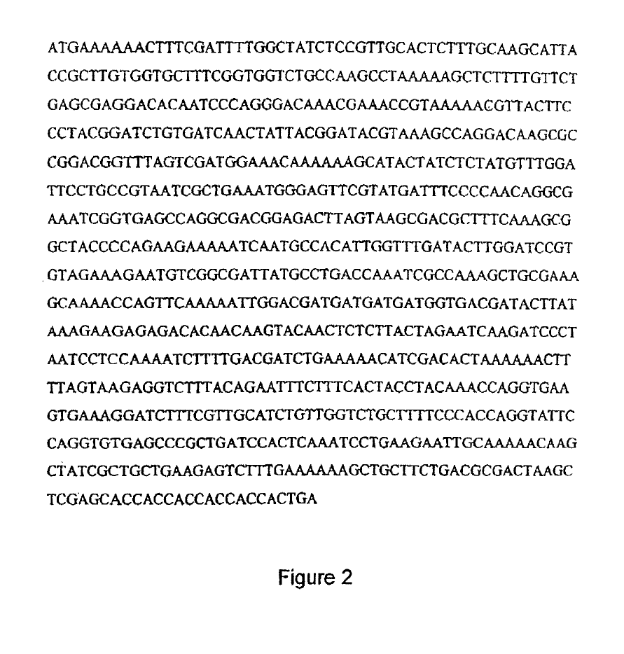

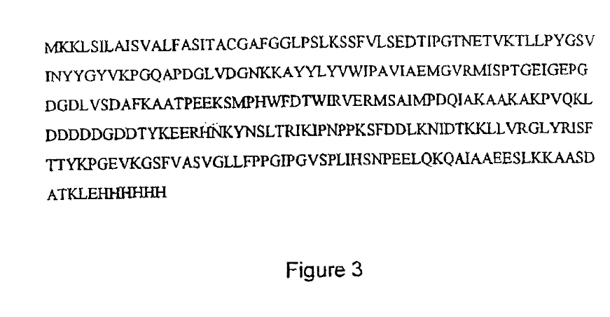

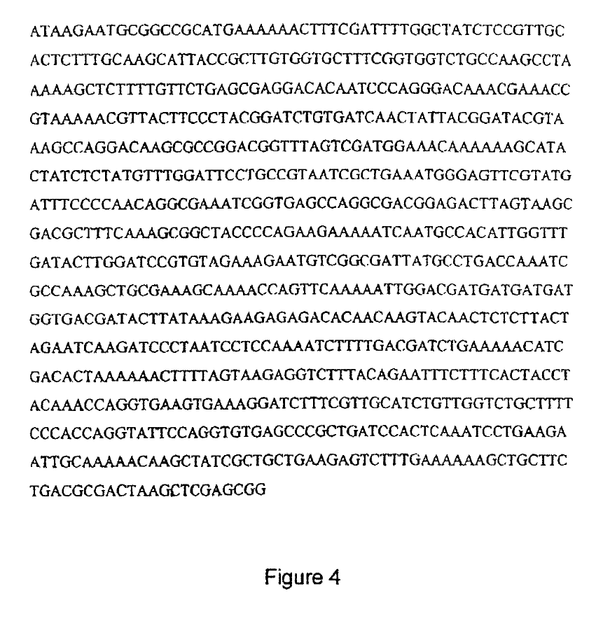

The PCR made it possible to obtain an 841 bp amplification product (SEQ ID NO: 5, FIG. 4). The 841 bp amplification product was purified using the QIAquick PCR purification kit (Qiagen) and then digested with NotI and XhoI. This fragment was ligated with the plasmid pET-29b (Novagen, Inc., Madison, Wis.) digested beforehand with NotI and XhoI, so as to generate the plasmid identified as pET-29b-ppl (FIG. 1). Complete sequencing was carried out and a homology search was performed on EXPASY. The results obtained show that the ppl gene (with Histag) comprises 843 bp and the PPL protein, of 30.678 kDa, consists of 280 amino acids (SEQ ID NO: 6 and 7, FIGS. 2 and 3).

The primers 1 (residues 17-39) and 2 (SEQ ID NO: 8) described above were tested on the genomic DNA of leptospires derived from the two species (sl): saprophytic (serotype patoc) and pathogenic (sl), in order to verify their specificity for pathogenic species. With regard to the pathogenic species, 7 genomic species were studied, including 6 pathogenic genospecies:

| genomic species | serotype | strain |
|---|---|---|
| weilii | celledoni | celledoni |
| kirshneri | cynopteri | 3522C |
| borgpetersenii | veldrat bataviae | 46 jav |
| noguchii | panama | CZ214K |
| santarosai | shermani | LT821 |
| interrogans sensu | autumnalis | S32 |
| stricto | | |
| interrogans sensu | icterohaemorrhagiae | RGA |
| stricto | | |
| biflexa sensu stricto | patoc | patoc |

Primers, identified at the Institut Pasteur, specific for the Leptospira gene were used as amplification controls.

No PCR product of approximately 800 bp was obtained for the genomic DNA derived from the saprophytic species or from the genomic species weilii.

These results, and those previously found, show that PPL is a protein specific for pathogenic leptospires.

A nucleotide sequence encoding the PPL protein was then incorporated into vectors (plasmid or viral vectors), so as to allow the production of the polypeptides of the invention in vitro or in vivo.

3.2. Prokaryotic Expression Vector and Production of the Protein in vitro:

The plasmid vector Pet 29b, which allows cloning in a multisite and expression of recombinant proteins in Escherichia coli (strain BL21 DE3), was chosen. The target gene was cloned into the plasmid under the control of a prokaryotic promoter inducible with isopropyl-β-thiogalactopyranoside (IPTG). The presence of a polyhistidine sequence (“Histag”) upstream of the multisite allowed detection and purification of the recombinant protein.

The vector Pet 29b-ppl was amplified in the Escherichia coli strain BL21(DE3) (Novagen Inc., Madison, Wis.).

The recombinant protein produced can be demonstrated with two types of antibody:

an anti-histidine antibody directed against the “Histag” of the protein at the C-terminal;

an anti-leptospire antibody originating from a rabbit immunized with live leptospires of the serogroup Autumnalis.

We sought to produce the recombinant protein in large quantity. The recombinant PPL protein was produced under standard conditions with an induction time of 180 minutes. The results obtained show the production of a recombinant protein of molecular mass 32 kDa, the purification of which was carried out as follows.

The purification, developed on polyhistidine columns (nickel: HiTrap™ Chelating, Amersham Pharmacia Biotech), was carried out using the FPLC (Fast Protein Liquid Chromatography) system in order to increase the yield and increase the purity of the recombinant protein produced.

3.3. Viral Vector and Production of the Protein in vivo

The invention allows the use of antibodies or polypeptides produced in vitro, with the aim of inducing an immune response and protection. This example shows, moreover, that an immune response can be obtained by expressing a nucleic acid of the invention in vivo, for example in a viral vector.

A nucleic acid encoding a PPL polypeptide of the invention was inserted into a nonreplicative human adenovirus (in this example, an adenovirus of the Ad5 type, defective for the E1 region) and a test for protection, with these recombinant viruses, against a challenge was carried out.

Preparation of the Adenoviruses

The plasmid pET-29b-ppl was digested with SalI and NaeI and the 1133 bp fragment was isolated after agarose gel electrophoresis. This fragment was then ligated with the plasmid pAd5CMV-linkintron which was first digested with NotI and then treated with T4 DNA polymerase (to make it blunt-ended), and then digested with SalI. The plasmid obtained was then digested with NheI and HindIII, then treated with T4 DNA polymerase and, finally, religated on itself. This construction makes it possible to introduce the ppl gene, the CMV (cytomegalovirus) promoter, a chimeric intron and the SV40 polyadenylation site.

The latter plasmid was digested with KpstI and NsiI; the 4 kb fragment was isolated after agarose gel electrophoresis and cotransformed into competent BJ5183 bacteria with a plasmid containing the defective human adenovirus serotype 5 genome.

This cotransformation made it possible to generate, by homologous recombination in Escherichia coli, a plasmid containing the defective human adenovirus serotype 5 genome and the ppl gene. This plasmid was designated pAd5-ppl.

293-cells are transfected, in a 6-well plate, at a density of 60 to 80% confluence, with 2 μg of pAd5-ppl digested beforehand with PacI, diluted in the presence of 10 μl of lipofectAMINE.

After transfection, the cells are left in culture for a few days until the appearance of a viral cytopathic effect (CPE). The cells are then again subcultured in order to amplify the recombined virus obtained. This virus is then cloned.

The recombined virus obtained is cultured and amplified in 293-cell culture. When the CPE is complete, the cells are harvested and lysed with 3 cycles of freezing-thawing, and the viral suspension is then clarified by low-speed centrifugation. The virus Ad-ppl is then purified on a cesium gradient (1.34 g/ml) and the viral band is harvested.

Desalification is carried out on PD10 columns (Pharmacia 51-1308-00); the virus is recovered in PBS, aliquoted and stored at −80° C. (with 10% glycerol). The virus is then titered and subjected to an RCA (replication competent adenovirus) test.

4. In vivo Protection Test

This example shows that the vectors of the invention can be used to induce a heterologous immune response in vivo.

4.1. First Protection Test

The test for protection, with the recombinant viruses, against a challenge was carried out under the following conditions.

A vaccination/challenge protocol was carried out in order to evaluate the protection which may be provided by a suspension of the recombined virus Ad-ppl which expresses the recombinant PPL protein (also designated P32). Two injections of 10⁹ CPED50 of Ad-ppl in a volume of 50 μl were given intramuscularly, three weeks apart. The challenge was performed two weeks later.

PPL batch: 15 animals

Absolute controls: 17 animals

After 21 days for the challenge (virulent strain canicola), survival was as follows:

PPL batch: 13/15 animals

Absolute controls: 8/17 animals.

These results clearly show that the PPL protein induces significant protection. It was identified from a strain of the serogroup Autumnalis and its gene made it possible to induce protection against a challenge performed with a strain belonging to the serogroup Canicola.

A vaccination/challenge protocol was also carried out in order to evaluate the protection which may be provided by the recombinant PPL protein. This protein is administered subcutaneously with an adjuvant. Three injections each containing 20 μg of recombinant PPL protein in a volume of 500 μl were administered at two-week intervals. The challenge was performed two weeks after the final injection. The results obtained confirm the immunogenicity of the polypeptides of the invention.

These results demonstrate that while the lipopolysaccharide antigens are clearly the carrier of effective protection against a homologous infection, which is the starting point for constituting the vaccinal preparations currently used both in humans and in animals, the PPL protein, used alone, is capable of inducing cross protection between different serogroups of the species Leptospira interrogans si (Icterohaemorrhagiae, Canicola, Autumnalis). Heterologous protection can also be induced between different genomic species of the traditional species Leptospira interrogans sl (here L. borgpetersenii and interrogans ss), whereas it is absent from the traditional saprophytic species Leptospira biflexa.

4.2. Vaccination with the Replication-Defective Recombinant Adenovirus

The aim of the vaccination with the live vectors is to analyze both the antibody response and the protection provided by the recombinant adenoviruses containing the ppl or ompL1 transgene.

4.2.1. Cloning the ppl Gene and ompL1 Gene (for Comparison) into a Eukaryotic Expression Vector

The cloning of the ppl gene was described previously.

The OmpL1 protein is a leptospire membrane protein studied by the team of Haake (Shang et al. 1993, Haake et al., 1995) and capable of providing protection against a homologous leptospire challenge (Haake et al., 1999).

The recombinant adenovirus Ad-ompL1 was constructed in the same way as that for the recombinant adenovirus Ad-ppl. The PCR primers were chosen so as to obtain the mature protein (without signal peptide).

4.2.2. Protocol for Vaccinating Gerbils with the Recombinant Viruses Ad-ppl and Ad-ompL1:

A vaccination/challenge protocol was carried out in order to evaluate the protection induced by a suspension of the recombinant virus Ad-ppl and Ad-ompL1, which express, respectively, the recombinant PPL protein and the recombinant OmpL1 protein.

Batch 1: 15 gerbils, 2 injections of 10⁹ CPED50 of adenovirus insert ppl in 50 μl

Batch 2: 15 gerbils, 2 injections of 10⁹ CPED50 of adenovirus insert ompL1 in 50 μl

Batch 3: 15 gerbils, 2 injections of 10⁹ CPED50 of adenovirus insert ppl+adenovirus insert ompL1 in 50 μl

Batch 4: 17 gerbils, 2 injections of PBS in 50 μl

Two injections of 10⁹ PFU of recombinant adenovirus in a volume of 50 μl were administered intramuscularly three weeks apart. The challenge was performed intraperitoneally two weeks later: Challenge canicola 10⁻⁵.

4.2.3. Results of Experiment 1

The results of the experiment in animals are given in the table below:

| | | | Batch 3 | |
|---|---|---|---|---|
| | Batch 1 | Batch 2 | Ad − ppl + | Batch 4 |
| Date | Ad −pp1 | Ad − ompL1 | Ad − ompL1 | Te(PBS) |
| Sep. 22, 1999 | 0/15 | 0/15 | 0/15 | 17 |
| Sep. 28, 1999 | 0/15 | 0/15 | 1/15 | 16 |
| Challenge: Can,d0 | 15 | 15 | 14 | 16 |
| d8 | 0/15 | 0/15 | 0/14 | 0/16 |
| d9 | 0/15 | 2/15 | 0/14 | 0/16 |
| d10 | 0/15 | 2/13 | 2/14 | 3/16 |
| d11 | 0/15 | 5/11 | 2/12 | 2/13 |
| d12 | 2/15 | 0/6 | 1/10 | 2/11 |
| d13 | 0/13 | 1/6 | 0/9 | 0/9 |
| d14 | 0/13 | 0/5 | 0/9 | 1/9 |
| d15 | 0/13 | 0/5 | 0/9 | 0/8 |
| Mortality rate | 13.4 | 66.7 | 35.7 | 50 |
| Survival rate | 86.6 | 33.3 | 64.3 | 50 |

The highest survival rate is obtained in batch 1, i.e. gerbils vaccinated with the recombinant adenovirus expressing the PPL protein (13/15). On the other hand, the mortality of batch 2, corresponding to the batch vaccinated with the adenovirus expressing the OmpL1 protein, is the highest of all groups (5/15) and in particular higher than the control group (8/16). It will also be noted that this mortality is earlier than in the other groups (from the ninth day after challenge).

This experiment makes it possible to demonstrate in a significant manner a protective effect of the PPL protein and the lack of protective effect of the OmpL1 protein.

With regard to the vaccination with two adenoviruses, an “intermediate” mortality between batch 1 and batch 2 is obtained (9/14).

There is no synergistic effect of the two recombinant proteins on the protection against a canicola leptospire challenge.

4.2.4. Validation of Results

The experiment consisting of vaccination with a live vector (replication-defective human adenovirus serotype 5 in 3.3) was carried out again under the same conditions in order to validate the results. However, it appeared to us to be more advantageous to use Ad5 without a transgene, instead of PBS, as a negative control, in order to determine the possible effect of the adenovirus in the protective response.

The results were as follows:

| | Batch 1 | Batch 2 | Batch 3 | Batch 4 |
|---|---|---|---|---|
| | Ad − pp1 | Ad − ompL1 | Ad − ppl + Ad − ompL1 | Te (Ad) |
| Challenge: Can,d0 | 15 | 15 | 15 | 17 |
| d8 | 0/15 | 0/15 | 0/15 | 0/17 |
| d9 | 0/15 | 1/15 | 0/15 | 0/17 |
| d10 | 0/15 | 1/14 | 0/15 | 1/17 |
| d11 | 1/15 | 1/13 | 0/15 | 2/16 |
| d12 | 0/14 | 2/11 | 1/15 | 0/14 |
| d13 | 1/14 | 1/10 | 1/14 | 1/14 |
| d14 | 0/13 | 2/9 | 0/13 | 2/13 |
| d15 | 0/13 | 0/7 | 0/13 | 0/11 |
| d16 | 0/13 | 1/7 | 0/13 | 2/11 |
| d17 | 0/13 | 1/6 | 0/13 | 0/9 |
| Mortality rate | 13.3 | 60 | 13.3 | 47.1 |
| Survival rate | 86.6 | 40 | 86.6 | 52.9 |

The mortality in the controls is similar to experiment 1: close to the lethal dose of 50.

This test shows that the adenovirus is not responsible for the difference in the survival rates.

These results are entirely comparable to the previous experiment and confirm the protective effect of PPL expressed by the replication-defective human adenovirus serotype 5. With regard to the vaccination with the recombinant OmpL1 protein expressed by the defective adenovirus type 5, results comparable to experiment 1 were obtained: highest mortality (6/15) and earliest mortality (d9).

4.2.5. Statistical Analysis of the Results from the Two Experiments

Statistical analysis of the tests of vaccinations
with the adenovirus, according to the Logrank test

| | Experiment 1 | | Experiment 2 | | total | |
|---|---|---|---|---|---|---|
| | χ² | ρ | χ² | ρ | χ² | ρ |
| Ad-ppl/control | 6.64 | <1.10⁻² | 5.52 | <2.10⁻² | 10.77 | <5.10⁻³ |
| Ad-ppl/Ad-ompL1 | 8.9 | <5.10⁻³ | 9.22 | <5.10⁻³ | 14.35 | <1.10⁻³ |
| ad-ppl + Ad-ompL1/control | 1.34 | NS^{a} | 8.54 | <5.10⁻³ | 7.56 | <1.10⁻² |
| Ad-ppl + Ad-ompL1/AdompL1 | 5.48 | <2.10⁻² | 8.62 | <5.10⁻³ | 9.91 | <5.10⁻³ |
| Ad-ompL1/control | 4.59 | <5.10⁻² | 3.87 | <5.10⁻² | 7.48 | <1.10⁻² |

The recombinant PPL protein expressed by the defective adenovirus type 5 provides significant protection against a canicola challenge for the two experiments. This protection is significant whatever the experiment, and combination of the two experiments increases the significance of the experiment, whether with the χ² test or the Logrank test.

The recombinant OmpL1 protein expressed by the defective adenovirus type 5 provides no protection. In addition, the Logrank test makes it possible to show, in a significant manner, that this recombinant protein, mediated by the adenovirus, has a negative effect on the survival rate for the two experiments.

4.3. Conclusion

Vaccination with the recombinant adenovirus made it possible to show the protective effect of the PPL protein, twice, subsequent to a heterologous (canicola) challenge.

5. Production of Anti-PPL Monoclonal Antibodies

5.1 Aims

The production of anti-PPL monoclonal antibodies corresponds to several aims:

to have a very reliable detection tool

to prepare affinity columns with these monoclonal antibodies in order to be able to obtain the native PPL protein from leptospire extracts

to test the possible passive immunization induced by these monoclonal antibodies.

5.2 Results

5.2.1. Production of Eight Monoclonal Antibodies

The monoclonal antibody production was carried out according to the conventional protocol (Maniatis). Eight monoclonal antibodies were obtained and then tested on:

total protein extracts of various pathogenic serogroups

total protein extracts of a saprophytic leptospire serogroup

the recombinant PPL protein

These experiments made it possible to show the production of 3 types of monoclonal antibody recognizing one, two or 3 bands by Western blotting on the various pathogenic leptospire extracts and the recombinant PPL protein (this confirms the results obtained with the anti-histidine antibody and the anti-leptospire rabbit antibody on the recombinant protein). Only one of the 8 monoclonal antibodies recognizes only one band on a nonpathogenic extract. The 3 bands recognized by Western blotting may correspond to a difference in the maturation stage of the protein or to a different conformation. Consequently, a monoclonal antibody which recognizes these 3 bands is an antibody which recognizes an epitope which is present whatever the maturation stage of the protein or which is accessible whatever the conformation of the protein. Thus, for passive immunization, a monoclonal antibody which is both absent in saprophytic leptospires and recognizes 3 bands by Western blotting is the best candidate.

5.2.2. Passive Immunization

The monoclonal antibody chosen for the passive immunization, corresponding to the criteria mentioned above, is the monoclonal antibody 6E5A4F2.

Protocol for the passive immunization

On D0, the gerbils from 3 batches received, intraperitoneally, a canicola challenge at 10⁻³ (1/10 dilution of the 10⁻² dilution made in EMJH base with 0.1% azide corresponding to the concentration existing in the culture supernatant containing the monoclonal 6E5A2F2).

On D1, batches 1 and 2 receive an injection of 0.5 ml of the culture supernatant containing the monoclonal antibody 6E5A2F2.

On D2, batch 2 receives a further injection of 0.5 ml of the culture supernatant containing the monoclonal antibody 6E5A2F2.

These two injections are given subcutaneously.

The results of the experiment in animals are given in the table below:

| | | | Batch 2 | | | |
|---|---|---|---|---|---|---|
| Challenge: Can, d0 | Batch 1 (d1) | | (d1 and d2) | | Batch 3: ct | |
| sc inj. of 0.5 | 9 animals | | 9 animals | | 9 animals | |
| ml of Mab | dead | alive | dead | alive | dead | alive |
| d7 | 0 | 9 | 0 | 9 | 3 | 6 |
| d8 | 0 | 9 | 0 | 9 | 1 | 5 |
| d9 | 0 | 9 | 0 | 9 | 1 | 4 |
| d10 | 0 | 9 | 0 | 9 | 1 | 3 |
| d11 | 2 | 7 | 0 | 9 | 0 | 3 |
| d12 | 0 | 7 | 1 | 8 | 0 | 3 |

| | | | |
|---|---|---|---|
| alive/number | 7/9 | 8/9 | 3/9 |
| Mortality as % | 22.2% | 11.1 | 66.7 |
| Alive as % | 87.8% | 88.9 | 33.3 |

5.3 Conclusion

The results are significant according to the χ² test. It is noted that the mortality in the control batch is high. Analysis of the table shows that the mortality in the two batches treated with monoclonal antibody 6E5A2F2 is very low (1/9 and 2/9) and later than in the control batch. Passive protection with the monoclonal antibody 6E5A2F2 is therefore demonstrated.

These results are in agreement with the results obtained previously with the vaccination with the adenovirus.

6. Characterization of the PPL Protein

6.1 Aim

The aim is to test the hemolytic activity of the recombinant PPL protein.

6.2 Hemolytic Activity of the Recombinant PPL Protein

Protocol

Test 1

The hemolytic activity of the recombinant PPL protein was tested, initially, on canine and human erythrocytes (group O).

Blood samples from dogs and humans were centrifuged at 2500 g for 3 minutes. The pellet of red blood cells is taken up in 4 to 5 ml of physiological saline. This step is repeated three times. A further centrifugation (2500 g, 3 min) makes it possible to recover the pellet of red blood cells, which is diluted so as to obtain a concentration of 4%.

60 μl of the suspension of canine or human erythrocytes at 4% are brought into contact with 60 μl of recombinant PPL protein (1.5 mg/ml) at various concentrations (serial dilutions), for 1 hour at 37° C. After centrifugation at 2500 g for 3 min, the supernatants are recovered.

The concentration of hemoglobin released into the supernatants was read by optical density (OD) at 540 nm.

During this study, a positive control, prepared in the presence of 1% Triton X100 (nonionic detergent), which corresponds to 100% release of hemoglobin, and a negative control (of PBS) were added.

Test 2

A second test, regarding only canine erythrocytes, was undertaken. The erythrocyte preparation is identical, but at a concentration of 2%. 50 μl of the preparation of erythrocytes at 2% are brought into contact with 100 μl of the recombinant PPL protein (1.5 mg/ml) at various concentrations (serial dilutions), for 1 hour at 37° C. After centrifugation at 2500 g for 3 min, the supernatants are recovered. The concentration of hemoglobin released into the supernatants was read by optical density (OD) at 540 nm. 1% Triton X100 and PBS were used as positive and negative control, respectively.

Results

Test 1

| | Dog red blood | Human red blood |
|---|---|---|
| | cells OD | cells OD |
| 75 μg PPL | 0.095 | 0.006 |
| 1% Triton X100 | 1.170 | 0.870 |
| PBS | 0.001 | 0 |

The canine erythrocytes appear to be sensitive to the hemolytic activity of the PPL protein. However, a further test in which the concentration of the protein is increased and the concentration of red blood cells is decreased is essential.

No hemolytic activity of the recombinant PPL protein is detected on the human red blood cells, under identical conditions. The human erythrocytes are therefore less sensitive than the canine erythrocytes to the activity of recombinant PPL protein.

Test 2

| | concentration | | |
|---|---|---|---|
| | pure | ½ dilution | ¼ dilution |
| OD recombinant PPL | 0.271 | 0.181 | 0.015 |
| OD 1% Triton X100 | 0.335 | 0.357 | 0.359 |
| OD PBS | 0 | — | — |

The hemolytic activity of the recombinant PPL protein on canine erythrocytes is demonstrated. This activity depends on the concentration of the recombinant PPL protein.

Conclusion

The recombinant PPL protein has hemolytic activity.

1. A polypeptide comprising the sequence SEQ ID NO: 1.

2. The polypeptide as claimed in claim 1, characterized in that it is a leptospire protein, in particular a (membrane and secreted) leptospire protein, more preferentially a pathogenic leptospiral strain.

3. A PPL protein, having a molecular weight of approximately 32 kD and comprising the sequence SEQ ID NO: 7.

4. A polypeptide or peptide, characterized in that it comprises the sequence of a protein originating from a pathogenic leptospire, in particular of a membrane or secreted protein of a pathogenic leptospire, or of a region of such a protein, and in that it bears one or more epitopes which are immunogenic with respect to several pathogenic leptospiral strains.

5. An immunogenic fragment of a protein or of a polypeptide as claimed in one of claims 1 to 4.

6. An antibody which recognizes a protein, a polypeptide or a peptide as claimed in one of claims 1 to 5.

7. The antibody as claimed in claim 6, characterized in that it is specific for pathogenic leptospires.

8. The antibody as claimed in claim 6 or 7, characterized in that it is a polyclonal antibody prepared by immunizing an animal with a protein, a polypeptide or a peptide as claimed in one of claims 1 to 5.

9. The antibody as claimed in claim 6 or 7, characterized in that it is a monoclonal antibody.

10. The antibody as claimed in one of claims 6 to 9, characterized in that it recognizes the PPL protein as claimed in claim 3 or a fragment thereof.

11. The antibody as claimed in one of claims 6 to 10, characterized in that it recognizes at least two pathogenic leptospiral strains.

12. The antibody as claimed in one of claims 6 to 11, characterized in that it recognizes the PPL protein at various maturation stages.

13. The antibody as claimed in claim 12, characterized in that it does not recognize saprophytic leptospires.

14. A fragment or derivative of an antibody as claimed in one of claims 6 to 13.

15. A nucleic acid encoding a polypeptide, peptide or protein as claimed in one of claims 1 to 5.

16. A vector comprising a nucleic acid as claimed in claim 15.

17. The vector as claimed in claim 16, characterized in that it is a plasmid or a recombinant virus.

18. A nucleotide probe and/or primer, which can be used for detecting and/or amplifying leptospire nucleic acids, and in particular for detecting the presence of a pathogenic leptospiral strain in a test sample, comprising all or part of a nucleic acid as claimed in claim 15.

19. The probe as claimed in claim 18, characterized in that it is single-stranded and in that it is labeled.

20. The probe as claimed in claim 18 or 19, characterized in that it is specific for pathogenic leptospiral strains and reacts with different serotypes and different genospecies of pathogenic leptospires.

21. The nucleotide primer as claimed in claim 18, characterized in that it is less than 40 bases in length, and in that it comprises the sequence of at least part of a nucleic acid as claimed in claim 15.

22. A pair of primers which allows a region of the gene encoding the PPL protein as claimed in claim 3 to be amplified.

23. The use of a polypeptide, peptide or protein as claimed in one of claims 1 to 5, for detecting the presence of anti-leptospire antibodies in a test sample.

24. A method for detecting the presence of anti-leptospire antibodies in a test sample, comprising bringing this sample (or a dilution) into contact with a polypeptide, peptide or protein as claimed in one of claims 1 to 5, and demonstrating the formation of antigen-antibody complexes.

25. The use of an antibody (or antibody fragment or derivative) as claimed in one of claims 6 to 14, for detecting the presence of a pathogenic leptospiral strain, or of a product secreted by said strain, in a test sample.

26. A method for detecting the presence of a pathogenic leptospiral strain, or of a product secreted by said strain, in a test sample, comprising bringing this sample (or a dilution) into contact with an antibody (or antibody fragment or derivative) as claimed in one of claims 6 to 14, and demonstrating the formation of antigen-antibody complexes.

27. A method for detecting (or screening for) the presence of a pathogenic leptospiral strain, or of a leptospire DNA fragment, in a test sample, comprising bringing the sample into contact with a nucleotide probe as claimed in one of claims 18 to 20, and demonstrating hybridization between said probe and said sample.

28. The method as claimed in claim 27, characterized in that the sample is treated beforehand so as to make the nucleic acids which it contains accessible to a hybridization reaction.

29. An immunogenic composition, comprising one or more polypeptides, peptides, proteins, antibodies, nucleic acids or vectors as claimed in one of claims 1 to 17.

30. The use of one or more polypeptides, peptides, proteins, antibodies, nucleic acids or vectors as claimed in one of claims 1 to 17, for preparing an immunogenic composition intended to induce a protective immune response against several leptospiral serotypes.

31. An immunogenic or vaccinal composition, comprising a polypeptide as claimed in one of claims 1 to 5 or a nucleic acid as claimed in claim 15.

32. A composition, comprising an antibody as claimed in one of claims 6 to 14.

33. A method for producing a polypeptide, peptide or protein as claimed in one of claims 1 to 5, comprising expressing a nucleic acid as claimed in claim 15 in a cell or organism, and recovering the polypeptide, peptide or protein produced.

34. A recombinant virus comprising a nucleic acid as claimed in claim 13.


SEQUENCE LISTING (8 sequences)

SEQ ID NO: 1 - protein, 16 aa, Leptospria sp.
TFLPYGSVINYYGYVK

SEQ ID NO: 2 - DNA, 28 bp, Artificial Sequence
  Oligonucleotide
gtnathaaytaytayggntaygtnaaar

SEQ ID NO: 3 - DNA, 39 bp, Artificial Sequence
  Oligonucleotide
ataagaatgcggccgcatgaaaaaactttcgattttggc

SEQ ID NO: 4 - DNA, 30 bp, Artificial Sequence
  Oligonucleotide
ccgctcgagcttagtcgcgtcagaagcagc

SEQ ID NO: 5 - DNA, 841 bp, Leptospira sp.
ataagaatgcggccgcatgaaaaaactttcgattttggctatctccgttgcactctttgcaagcattaccgcttgtggtgctttcggtggtctgccaagcctaaaaagctcttttgttctgagcgaggacacaatcccagggacaaacgaaaccgtaaaaacgttacttccctacggatctgtgatcaactattacggatacgtaaagccaggacaagcgccggacggtttagtcgatggaaacaaaaaagcatactatctctatgtttggattcctgccgtaatcgctgaaatgggagttcgtatgatttccccaacaggcgaaatcggtgagccaggcgacggagacttagtaagcgacgctttcaaagcggctaccccagaagaaaaatcaatgccacattggtttgatacttggatccgtgtagaaagaatgtcggcgattatgcctgaccaaatcgccaaagctgcgaaagcaaaaccagttcaaaaattggacgatgatgatgatggtgacgatacttataaagaagagagacacaacaagtacaactctcttactagaatcaagatccctaatcctccaaaatcttttgacgatctgaaaaacatcgacactaaaaaacttttagtaagaggtctttacagaatttctttcactacctacaaaccaggtgaagtgaaaggatctttcgttgcatctgttggtctgcttttcccaccaggtattccaggtgtgagcccgctgatccactcaaatcctgaagaattgcaaaaacaagctatcgctgctgaagagtctttgaaaaaagctgcttctgacgcgactaagctcgagcgg

SEQ ID NO: 6 - DNA, 843 bp, Leptospira sp.
  CDS (1)...(843)
atgaaaaaactttcgattttggctatctccgttgcactctttgcaagcattaccgcttgtggtgctttcggtggtctgccaagcctaaaaagctcttttgttctgagcgaggacacaatcccagggacaaacgaaaccgtaaaaacgttacttccctacggatctgtgatcaactattacggatacgtaaagccaggacaagcgccggacggtttagtcgatggaaacaaaaaagcatactatctctatgtttggattcctgccgtaatcgctgaaatgggagttcgtatgatttccccaacaggcgaaatcggtgagccaggcgacggagacttagtaagcgacgctttcaaagcggctaccccagaagaaaaatcaatgccacattggtttgatacttggatccgtgtagaaagaatgtcggcgattatgcctgaccaaatcgccaaagctgcgaaagcaaaaccagttcaaaaattggacgatgatgatgatggtgacgatacttataaagaagagagacacaacaagtacaactctcttactagaatcaagatccctaatcctccaaaatcttttgacgatctgaaaaacatcgacactaaaaaacttttagtaagaggtctttacagaatttctttcactacctacaaaccaggtgaagtgaaaggatctttcgttgcatctgttggtctgcttttcccaccaggtattccaggtgtgagcccgctgatccactcaaatcctgaagaattgcaaaaacaagctatcgctgctgaagagtctttgaaaaaagctgcttctgacgcgactaagctcgagcaccaccaccaccaccactga

SEQ ID NO: 7 - protein, 280 aa, Leptospira sp.
MKKLSILAISVALFASITACGAFGGLPSLKSSFVLSEDTIPGTNETVKTLLPYGSVINYYGYVKPGQAPDGLVDGNKKAYYLYVWIPAVIAEMGVRMISPTGEIGEPGDGDLVSDAFKAATPEEKSMPHWFDTWIRVERMSAIMPDQIAKAAKAKPVQKLDDDDDGDDTYKEERHNKYNSLTRIKIPNPPKSFDDLKNIDTKKLLVRGLYRISFTTYKPGEVKGSFVASVGLLFPPGIPGVSPLIHSNPEELQKQAIAAEESLKKAASDATKLEHHHHHH

SEQ ID NO: 8 - DNA, 19 bp, Artificial Sequence
  Oligonucleotide
ttacttagtcgcgtcagaa